(19) United States
(12) Patent Application Publication
(10) Pub. No.: US 2017/0152460 A1
(43) Pub. Date: Jun. 1, 2017

(54) MICROBIAL OIL AND FAT CLEANING AGENT COMPOSITION BLOCK FOR DRAINPIPES AND METHOD OF PRODUCING THE SAME

(71) Applicant: CHEONGSOO E&S INC., Seoul (KR)

(72) Inventors: Hee Pill LEE, Seoul (KR); Yong Bae KIM, Seoul (KR)

(73) Assignee: CHEONGSOO E&S INC., Seoul (KR)

(21) Appl. No.: 15/359,820

(22) Filed: Nov. 23, 2016

(30) Foreign Application Priority Data
Nov. 27, 2015 (KR) 10-2015-0167525
Feb. 23, 2016 (KR) 10-1598765

(51) Int. Cl. C11D 3/38; C11D 3/382; C12N 1/20; C11D 1/82; C11D 3/22; C11D 17/00; C11D 1/22; C11D 1/66

(52) U.S. Cl. CPC C11D 3/381; C11D 1/22; C11D 3/382; C11D 1/66; C11D 1/82; C11D 3/225; C11D 17/0047; C12N 1/20

(57) ABSTRACT

Microbial oil and fat cleaning agent composition block for drainpipes and a method of producing the same. Specifically, such a composition block accommodated in a drain cap for the inlet of a drainpipe so that oils and fats adhered to the drainpipe are biodegraded by powdery microorganisms present in the composition block to maintain smooth flow of sewage, prevent oil and fats from adhering onto the drainpipe from the cleaning agent, thus cleaning the inside of the drainpipe and removing odors. The dissolved cleaning agent is discharged with sewage through the drainpipe and is collected in a water collector, and oils and fats adhered to the water collector are biodegraded by the cleaning agent to remove odor inside the water collector, prevent adhesion of oils and fats to the water collector and thus maintain excellent water quality.

### BACKGROUND OF THE INVENTION

### Field of the Invention

The present invention relates to a microbial oil and fat cleaning agent composition block for drainpipes and a method of producing the same. More specifically, the present invention relates to a microbial oil and fat cleaning agent composition block for drainpipes in which a solid microbial oil and fat cleaning agent composition block accommodated in a drain cap equipped in an inlet of a drainpipe is dissolved and introduced into the drainpipe so that oils and fats adhered to the drainpipe can be biodegraded by powdery microorganisms present in the microbial oil and fat cleaning agent composition block to maintain smooth flow of sewage, prevent oil and fats from adhering onto the drainpipe owing to the cleaning agent and thus clean the inside of the drainpipe and remove odors, and the dissolved microbial oil and fat cleaning agent is discharged together with sewage through the drainpipe and is collected in a water collector and oils and fats adhered to the water collector are biodegraded by the microbial oil and fat cleaning agent to remove the odor inside the water collector, prevent adhesion of oils and fats onto the water collector and thus maintain excellent water quality, and a method of producing the cleaning agent composition block.

Description of the Related Art

In general, domestic sewage is discharged through a drain and various contaminants contained in sewage are attached to the drainpipe connected to the drain, thus making drain flow unsmooth and allowing odor to spread from propagated various bacteria backward through the drainpipe.

Wastewater produced during daily life is treated by sewage purification plants, septic tanks and sewage and/or wastewater treatment plants equipped in houses or industries, sewage and/or wastewater treatment plants in agricultural industrial complexes or the like.

The origins of sewage and wastewater related to human life are restrooms, baths, kitchens and the like of homes, restaurants and the like. The amount of generated sewage is proportional to the amount of water used and the main sewage generation source is waster used for flush toilets and during daily life. The amount of generated sewage continuously increases with increase in the amounts of contaminants and water supplied. Since 2000, according to enforcement regulations on sewage, excrement and livestock wastewater, there is a restriction to 10 ppm, regardless of treatment capacity of sewage purification plants.

In addition, a great amount of water is required due to industrial development, the gravitation of population toward the cities and diversification of life patterns. In particular, recently, large-scale apartments are constructed and amounts of sewage are increasing locally in certain regions. Sewage mostly discharged from homes are moved to sewage treatment plants, are subjected to suitable treatment processes in sewage terminal treatment plants and are then discharged to discharge zones.

However, only a part of all sewage can be treated with some sewage terminal treatment plants currently constructed in big cities. In addition, sewage which contains great amounts of non-biodegradable oils and fats due to excessive use of cleaning agents, synthetic detergents and the like is discharged and there are great difficulties in efficient wastewater treatment by conventional sewage treatment methods.

Recently, meat consumption is gradually increasing with the change of Koreans' taste, the amounts of fats used in hotels, department stores, fast food restaurants, supermarkets, convenience stores (kitchen, fish and meat departments), and food production factories for cooking and producing cooked meat products are increasing and the amount of discharged waste fats is also increasing. The discharged waste fats are adhered to the surfaces of sewage pipes and grow while forming an oil film, thus causing breakage of sewage pipes and thus sewage transportation problems.

In addition, waste fats introduced into sewage treatment plants form oil films on the surface of water present in the sewage treatment plants, thus inhibiting oxygen delivery, and the waste fats adhered to the surfaces of treatment plants have negative effects on treatment efficiency. Waste fats released to natural water systems are almost not naturally degraded, thus causing odor problems to the surrounding environments and the possibility of civil complaints.

Unlike other environmental pollutants, waste oils and fats require pre-treatment for disposal in generation spots or conversion into minimally stable substances. Accordingly, conventional treatment of waste fats has been performed by physically and/or chemically removing fat ingredients using pressure flotation equipment, a fat separation tank or the like, and then treating the same by an active sludge method. However, the conventional method is inefficient because it requires a large area for the pre-treatment device and entails much efforts and costs to dispose the removed waste fats. Accordingly, biological treatment to improve operation efficiency and reduce operation costs based on accurate prediction of amounts of generated wastewater and contained fat ingredients was tried by a novel method. However, the biological treatment was predominantly conducted using foreign fat treatment products and has a high possibility of odor generation because the odor was very readily emitted before fats were degraded due to very low treatment efficiency and limitation on treatment capacity without large-scale trap devices. In addition, most biological treatment agents are produced from microorganism preparations, in particular, transformed microorganisms or foreign microorganisms and are thus possibly transformed into new forms and may readily disturb ecosystems. Accordingly, there is an urgent need for development of strains living in the same ecosystem. In addition, conventional grease traps produced and sold by kitchen equipment companies are simple physical collectors and have secondary pollution problems.

Meanwhile, in Korea, legal regulations on waste fat ingredients are based on the content of a normal-hexane extract substance. In a clean area, the content of the normal-hexane was set to 5 ppm or less and, in A and B certain areas, the content of the normal-hexane was set to 30 ppm or less. However, treatment was difficult within the legal regulation range with conventional treatment methods.

### SUMMARY OF THE INVENTION

Therefore, the present invention has been made in view of the above problems, and it is an object of the present invention to provide a microbial oil and fat cleaning agent composition block for drainpipes in which a solid microbial oil and fat cleaning agent composition block accommodated in a drain cap equipped in an inlet of a drainpipe is dissolved and introduced into the drainpipe so that oils and fats adhered to the drainpipe can be biodegraded by powdery microorganisms present in the microbial oil and fat cleaning agent composition block to maintain smooth flow of sewage, prevent oils and fats from adhering onto the drainpipe due to the cleaning agent and thus clean the inside of the drainpipe and remove odors, and the dissolved microbial oil and fat cleaning agent is discharged together with sewage through the drainpipe and is collected in a water collector, and oils and fats adhered to the water collector are biodegraded by a microbial oil and fat cleaning agent to remove the odor inside the water collector, prevent adhesion of oils and fats into the water collector and thus maintain excellent water quality, and a method of producing the cleaning agent composition block.

In accordance with an aspect of the present invention, the above and other objects can be accomplished by the provision of a method of producing a microbial oil and fat cleaning agent composition block for drainpipes, the method including elevating a temperature of a reaction tank to 85° C. by heating, adding a non-ionic surfactant to the heated reaction tank and stirring the same to completely dissolve the non-ionic surfactant, adding a linear alkyl benzene-based surfactant to the completely dissolved non-ionic surfactant and stirring the same until the linear alkyl benzene-based surfactant is completely dissolved, secondarily adding a linear alkyl benzene-based surfactant and stirring the same until the linear alkyl benzene-based surfactant is completely dissolved while maintaining a temperature at 80° C. or less, adding a non-ionic higher alcohol and stirring the same while maintaining a temperature at 75° C., adding a cleaning agent and stirring the same while maintaining a temperature at 75 to 76° C., adding a dye, stirring the same while maintaining a temperature at 65 to 70° C. and cooling the same, adding a dissolution controller and stirring the same, adding an organic microorganism powder and stirring the same while maintaining a temperature at 65 to 70° C., adding a thickener and a defoamer and stirring the same, and filling a die with the composition mixed in the previous steps.

Preferably, the organic microorganism powder may include bacillus strain.

In another aspect of the present invention, provided is a microbial oil and fat cleaning agent composition block for drainpipes which is produced by the method and includes 31 to 34% by weight of the non-ionic surfactant added during the initial addition of the non-ionic surfactant, 14 to 16% by weight of the linear alkyl benzene-based surfactant added during addition of the linear alkyl benzene-based surfactant, 15 to 17% by weight of the linear alkyl benzene-based surfactant added during secondary addition of the linear alkyl benzene-based surfactant, 15 to 17% by weight of the non-ionic higher alcohol, 7 to 9% by weight of the cleaning agent, 0.1% by weight of the dye, 4.5 to 5.5% by weight of the dissolution controller, 2.5 to 3.5% by weight of the organic microorganism powder, 3.5 to 4.5% by weight of the thickener, and 2.5 to 3.5% by weight of the defoamer.

Preferably, the organic microorganism powder may include bacillus strain.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flowchart sequentially illustrating steps of a method of producing a microbial oil and fat cleaning agent composition block for drainpipes according to an embodiment of the present invention;

FIG. 2 is an image comparing degradation of oils and fats between a tube to which the microbial oil and fat cleaning agent composition block for drainpipes according to an embodiment of the present invention is fed and a tube to which the cleaning agent composition is not fed under the same conditions over 15 days;

FIG. 3 is an image showing a beaker to which the same amount of the composition block is fed and then melted;

FIG. 4 is images comparing the inside of a drainpipe, the inside of a water collector and qualities of water present inside of the water collector before and after feeding of the microbial oil and fat cleaning agent composition block for drainpipes according to an embodiment of the present invention;

FIG. 5 is a test report showing microorganism measurement results of powdery microorganism samples;

FIG. 6 is a test report showing microorganism measurement results present in the microbial oil and fat cleaning agent composition block for drainpipes according to the present invention; and

FIG. 7 is another test report showing microorganism measurement results present in any block of a trial product sample according to the present invention.

FIG. 8 is a graph showing the cleaning action of domestic wastewater fed to a drainpipe by the bacillus strain.

### DETAILED DESCRIPTION OF THE INVENTION

Hereinafter, a method of producing a microbial oil and fat cleaning agent composition block for drainpipes and a microbial oil and fat cleaning agent composition block for drainpipes prepared by the method according to an embodiment of the present invention will be described with reference to the annexed drawings.

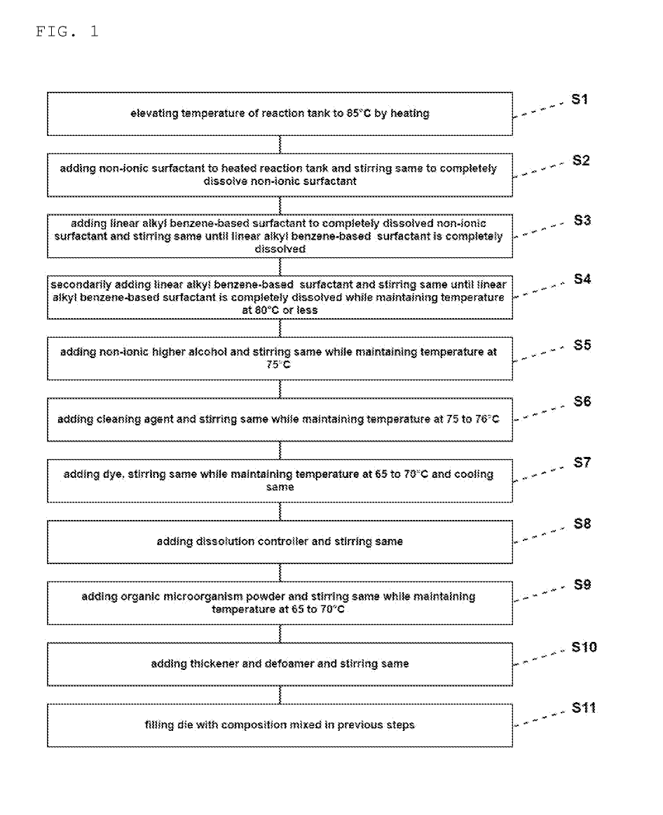

Regarding the method of preparing a microbial oil and fat cleaning agent composition block for drainpipes according to the embodiment of the present invention, as shown in FIG. 1 annexed herein, first, an inner temperature of a reaction tank for mixing an oil and fat cleaning agent composition with stirring was elevated to 85° C. by heating (S1). The inner temperature of the reaction tank of 85° C. is an optimal temperature which minimizes the viscosity of a non-ionic surfactant introduced and stirred in the subsequent step and thereby offers thorough dispersion and efficient stirring.

A non-ionic surfactant was added to the reaction tank heated to 85° C. and stirred at a stirring rate of 7.0 rpm for 10 to 30 minutes, preferably for 20 minutes, to completely dissolve the non-ionic surfactant (S2). The non-ionic surfactant is a surfactant which has advantages of free mixing ability with other surfactants or electrolytes, excellent foam stability, non-harmfulness of skin response, excellent viscosity improvement, good low-temperature stability, formation of less foams and excellent cleaning effects. In this step, the non-ionic surfactant is introduced in an amount of 31 to 34% by weight, based on the total weight of the solid microbial oil and fat cleaning agent for drainpipes according to the present invention. Examples of the non-ionic surfactant include, but are not limited to, polyoxyethylene alkyl ether, polyoxyethylene fatty acid ester, polyoxyethylene alkyl phenol ether, sorbitan fatty acid ester, polyoxyethylene sorbitan fatty acid ester and the like. Any non-ionic surfactant is preferably used so long as it is used in the cleaning agent field and is suitable for microorganisms.

After complete dissolution of the non-ionic surfactant, a linear alkyl benzene-based surfactant is added thereto and is stirred for about 30 to 40 minutes until it is completely dissolved (S3). The linear alkyl benzene-based surfactant includes alkylbenzene sulfonates (also called “LAS”), alcohol ethoxy sulfates, alpha-olefin sulfonates, fatty alkane amides and the like. When the linear alkyl benzene-based surfactant is dissolved in water, its hydrophilic group is dissociated into an anion, thus offering easy degradation using microorganisms and providing excellent cleaning capacity and foaming capacity while generating no contaminants. In this step, the linear alkyl benzene-based surfactant is introduced in an amount of 14 to 16% by weight, based on the total weight of the solid microbial oil and fat cleaning agent for drainpipes according to the present invention.

The linear alkyl benzene-based surfactant introduced during S3 is secondarily added to the mixed solution completely dissolved in S3 and is stirred until it is completely dissolved (S4). The reason for adding the linear alkyl benzene-based surfactant in S3 again in this step is that the surfactant is added in two portions (primarily and secondarily) to completely dissolve due to the high weight proportion of added surfactant and it is more suitable for complete dissolution since a dissolution temperature is gradually decreased as the added linear alkyl benzene-based surfactant is dissolved. The linear alkyl benzene-based surfactant added in the present step is the same as the linear alkyl benzene-based surfactant added in S3 and a detailed explanation thereof will be omitted. In the present step, stirring is conducted for 40 to 50 minutes for complete dissolution while the temperature is maintained at 80° C. or less. The reason for maintaining the inner temperature at 80° C. is to maintain a temperature suitable for dissolution of the linear alkyl benzene-based surfactant and survival of powdery microorganisms which will be added later. In this step, the linear alkyl benzene-based surfactant is introduced in an amount of 15 to 17% by weight, based on the total weight of the solid microbial oil and fat cleaning agent for drainpipes according to the present invention.

Next, a non-ionic higher alcohol is added and stirred at 75° C. (S5). In this step, stirring is conducted at a stirring rate of 7.0 rpm for 20 minutes. The non-ionic higher alcohol includes a polyhydric alcohol, has advantages of excellent moisturizing property and less skin troubles and is neutral and almost insoluble in water. As the non-ionic higher alcohol added in the present step, a non-ionic higher alcohol having 12 to 16 carbon atoms is used as a cleaning agent and detergent, a non-ionic higher alcohol having 12 to 16 carbon atoms is mainly used as a solvent or plasticizing agent, and a non-ionic higher alcohol having 16 or more carbon atoms is used for cosmetics and medicines. The reason for maintaining the temperature at 75° C. is to maintain the temperature suitable for survival of powdery microorganisms which will be added later. In this step, the higher alcohol is introduced in an amount of 15 to 17% by weight, based on the total weight of the solid microbial oil and fat cleaning agent for drainpipes according to the present invention.

A cleaning agent is added to the mixed solution obtained by stirring the mixed composition for complete dissolution and then stirred (S6). In the present step, the cleaning agent is stirred at a stirring rate of 9.5 rpm for 60 minutes while maintaining the temperature at 75 to 76° C. The reason for maintaining the temperature is to maintain a temperature suitable for survival of powdery microorganisms added later, like S4 and S5. In this step, the cleaning agent is introduced in an amount of 7 to 9% by weight, based on the total weight of the solid microbial oil and fat cleaning agent for drainpipes according to the present invention.

Next, in order to impart color to the microbial oil and fat cleaning agent for drainpipes according to the embodiment of the present invention, a dye is added and mixed with stirring and cooled (S7). In the present step, the stirring is conducted at a stirring rate of 9.5 rpm while maintaining the temperature at 65 to 70° C. and then cooled for 12 hours. In this step, the dye is introduced in an amount of 0.1% by weight, based on the total weight of the solid microbial oil and fat cleaning agent for drainpipes according to the present invention. The dye is preferably a common blue or navy blue dye.

After the dye cooling (S7), a dissolution controller is added (S8). The dissolution controller added in this step controls homogeneous dissolution of a surfactant, a cleaning agent or the like in the mixture. In this step, the dissolution controller is introduced in an amount of 4.5 to 5.5% by weight, based on the total weight of the solid microbial oil and fat cleaning agent for drainpipes according to the present invention.

Next, a powdery microorganism is added and stirred to homogeneously disperse the powdery microorganism in the solid mixture while maintaining the temperature at 65 to 70° C. (S9). The most important reason for maintaining the temperature at 65 to 70° C. is to obtain a liquid form and thereby maximize movement efficiency to improve survival probability of organic microorganisms. In this step, the organic microorganism powder is introduced in an amount of 2.5 to 3.5% by weight, based on the total weight of the microbial oil and fat cleaning agent for drainpipes according to the present invention. The organic microorganism powder for drainpipes according to the present invention is added three ⅓ batches and stirring time is 3 hours or longer so that the powdery microorganism is sufficiently homogeneously dispersed in the mixture.

The powdery microorganism added during this step is a highly-concentrated powder, is a bacillus strain which includes 6.0 to 7.0×10⁹ cfu/g bacteria, is also called “Bacillus subtillis”, is bacteria which degrade proteins present in beans during production of Cheonggukjang or Doenjang and is generally active under a neutral condition, i.e., pH 7.

The bacillus strain is a strain with excellent degradation and cleaning capacities which produces optimum enzymes by efficient degradation of cellulose, fats, proteins, carbohydrates and the like owing to superior active reactions of bacteria, removes odor based on germination and growth of bacteria, degrades oils and fats accommodated and coagulated on inner walls of drainpipes and inside the water collector, greatly inhibits an increase in ammonia concentration and performs washing and cleaning functions with the surfactant.

That is, fats, oils and grease commonly called “FOG” are degraded in such a manner that glycerin is separated and decomposed from fatty acid of oils and fats by cell degradation using fatty acid non-germ cell bacteria, i.e., bacillus strain. In this regard, the bacillus strain performs washing and cleaning functions with a surfactant.

In addition, the bacillus strain is bacteria which act under both aerobic and anaerobic conditions and act the most effectively at a pH of 5.0 to 10.0 and at a temperature of 5 to 55° C.

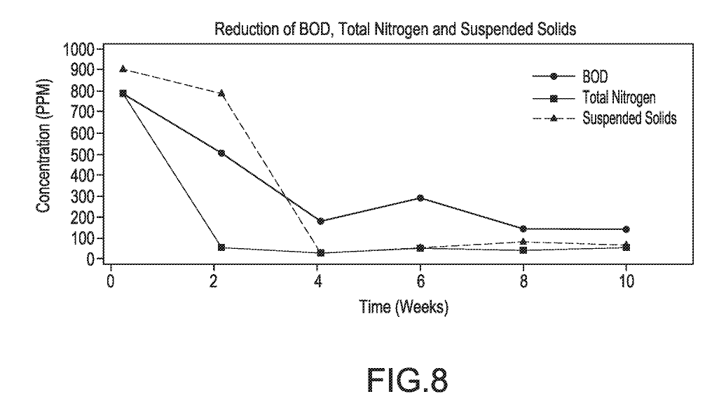

The cleaning action of domestic wastewater fed to the drainpipe by the bacillus strain is shown in FIG. 8. When the bacillus strain is fed, biochemical oxygen demand (BOD), total nitrogen and suspended solids of wastewater gradually decrease over time, in particular, total nitrogen and suspended solids rapidly decrease to 1/10 or less since 4 weeks and cleaning capacity thereof gradually increases after 4 weeks. In addition, suspended solids showed by a green graph rapidly decrease 2 to 4 weeks after introduction of the bacillus strain. As can be seen from Test Example described later, the bacillus strain is very effective in degradation of oils and fats introduced to the drainpipe.

When the bacillus strain is added, stirred and homogeneously dispersed in the solid mixture, in the final addition step, a thickener and a defoamer are added and stirred (S10). The thickener is a substance for increasing the viscosity of the mixture, is a pine oil and is added in an amount of 3.5 to 4.5% by weight, based on the total weight of the solid microbial oil and fat cleaning agent for drainpipes according to the present invention. The defoamer is a water-soluble surfactant used to remove foams and inhibit formation of foams with an oily substance having low volatility and high diffusion capacity. The defoamer used in this step is a silicon defoamer having broad applicability due to chemical stability and excellent defoaming activity and is added in an amount of 2.5 to 3.5% by weight, based on the total weight of the solid microbial oil and fat cleaning agent for drainpipes according to the present invention. The thickener and defoamer are added in this step 30 minutes immediately before the subsequent step (the filling step) which will be described layer, and are then stirred at a stirring rate of 5.0 rpm for 30 minutes.

In the final step, S10, a cylindrical die is filled with the mixture of the thickener and the defoamer, which had been added and stirred in S10, and is then cooled (S11) in order to create a shape accommodated in the drain cap equipped in the drainpipe, thereby producing a solid microbial oil and fat cleaning agent composition block for drainpipes having a predetermined shape according to the embodiment of the present invention. The die for filling used in this step is well-known in the art and a detailed explanation thereof will be omitted.

As described above, the microbial oil and fat cleaning agent composition block for drainpipes obtained by a method of producing the microbial oil and fat cleaning agent composition block for drainpipes according to the embodiment of the present invention including sequentially performing the steps of S1 to S11 includes 31 to 34% by weight of a non-ionic surfactant, 29 to 33% by weight of a linear alkyl benzene-based surfactant, 15 to 17% by weight of a non-ionic higher alcohol, 7 to 9% by weight of a cleaning agent, 0.1% by weight of a dye, 4.5 to 5.5% by weight of a dissolution controller, 2.5 to 3.5% by weight of an organic microorganism powder, 3.5 to 4.5% by weight of a thickener and 2.5 to 3.5% by weight of a defoamer, based on the total weight of the microbial oil and fat cleaning agent for drainpipes according to the present invention, and is a block-type microbial oil and fat cleaning agent composition for drainpipes which can maintain its shape after cooling.

The microbial oil and fat cleaning agent block for drainpipes according to the present invention is preferably produced in a unit amount of 40 g and in a unit amount of 100 g at maximum depending on user's demand.

### EXAMPLE 1

324 g of linoleic acid was added as a non-ionic surfactant to a reaction tank heated to 85° C. and stirred at a stirring rate of 7.0 rpm for 20 minutes. After complete dissolution, 30 g of a room temperature emulsifier (CME) was added and 115 g of linear alkylbenzene sodium sulfonate was primarily added and stirred for 30 minutes. 90 g of a room temperature emulsifier (CME) was secondarily added to the completely dissolved mixture and 60 g of linear alkylbenzene sodium sulfonate was then added thereto. The mixture was stirred for 40 minutes while maintaining the temperature at 80° C. or less. Then, 160 g of a higher alcohol cleaning agent was added as a non-ionic higher alcohol and was stirred at a stirring rate of 7.0 rpm at a constant temperature of 75° C. for 20 minutes. 80 g of a cleaning agent was added to the completely dissolved mixture solution and stirred at a temperature of 75 to 76° C. and at a stirring rate of 9.5 rpm for 60 minutes. Then, 1 g of a blue dye was added to the reaction solution at a temperature of 65 to 79° C. and cooled for 12 hours while stirring at a stirring rate of 9.5 rpm. 50 g of carboxymethyl cellulose was added as a dissolution controller to the cooled mixture solution, g of a powdery microorganism (Bacillus subtillis) was added three 10 g batches and the temperature was maintained at 67° C. while stirring for 3 hours. 30 minutes before filing a die with the reaction product, 30 g of an emulsion-type defoamer and 40 g of a pine oil as a thickener were added and stirred at a stirring rate of 5.0 rpm for 30 minutes and a die was filled with the mixture to obtain 1,000 g of a cleaning agent composition block.

### TEST EXAMPLE 1

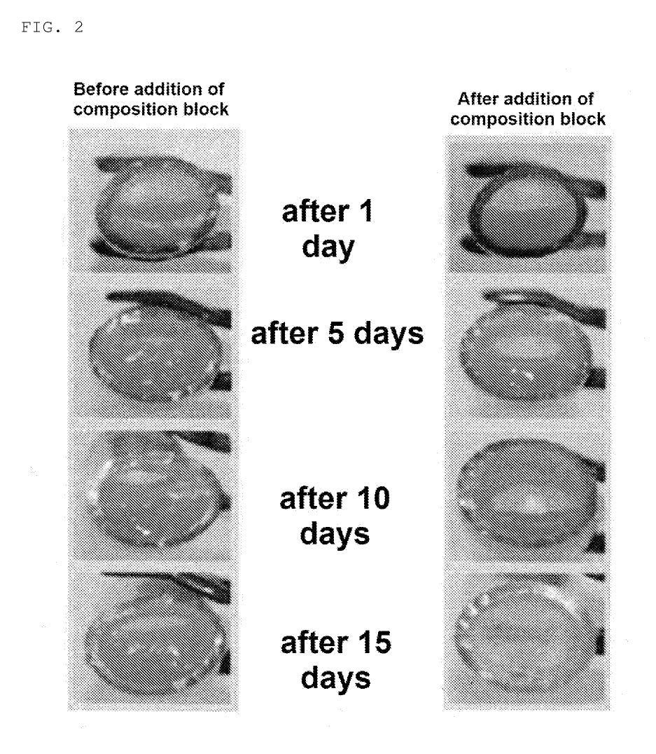

A test for comparing degradation degrees of oils and fats fed into a drainpipe under the following conditions was performed using the microbial oil and fat cleaning agent composition block for drainpipes obtained by the method of producing a microbial oil and fat cleaning agent composition block for drainpipes according to the embodiment of the present invention and results of Test Example will be described with reference to FIG. 2 annexed herein.

Test subject: identical weights of oils and fats produced with grease, butter, fat (purified and hardened), hardened coconut oil, powdery gelatin protein, sugar, egg yolk, red fat-soluble dye, water or the like.

Test equipment: plastic tube, tube cover (cork), stand, clamp, mass cylinder, spuit, beaker

Test method: identical weights of oils and fats produced by mixing test subjects such as grease, butter and fat were fed to a plastic tube, both ends were sealed with a stopper and the tube was cured in a freezer for 12 hours. Then, a powder obtained by grinding the microbial oil and fat cleaning agent composition block for drainpipes according to the present invention was fed into one tube and the tube was then sealed. On the other hand, the powder was not fed to another tube, the tube was sealed and compared by the naked eye at room temperature for 15 days.

Test results:

Regarding the tube to which the powder obtained by grinding the microbial oil and fat cleaning agent composition block for drainpipes according to the present invention is fed, as shown in the right image of the annexed FIG. 2, oils and fats each added on the 1^{st}, 5^{th}, 10^{th} and 15^{th} days were gradually degraded over time and about 80% of oils and fats were degraded after 15 days. On the other hand, regarding the tube to which only oils and fats were added without the microbial oil and fat cleaning agents for drainpipes according to the present invention, as shown in the left image of the annexed FIG. 2, oils and fats were not degraded even over time and the weight of oils and fats was thus almost not changed.

### TEST EXAMPLE 2

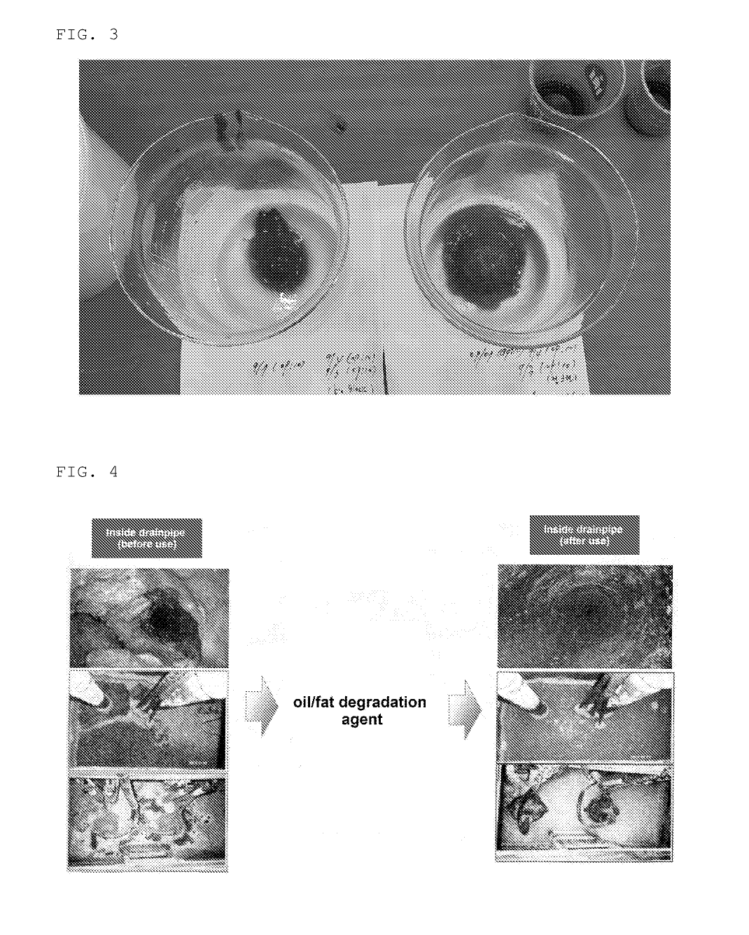

Identical weights of the microbial oil and fat cleaning agent composition block for drainpipes according to the present invention and another well-known composition block were added and used under the same conditions for 10 days. As a result, as shown in FIG. 3, the composition block according to the present invention was degraded and consumed more than the other well-known composition block (the left image of FIG. 3), which means that the composition block according to the present invention was more effective in cleaning oils and fats.

### TEST EXAMPLE 3

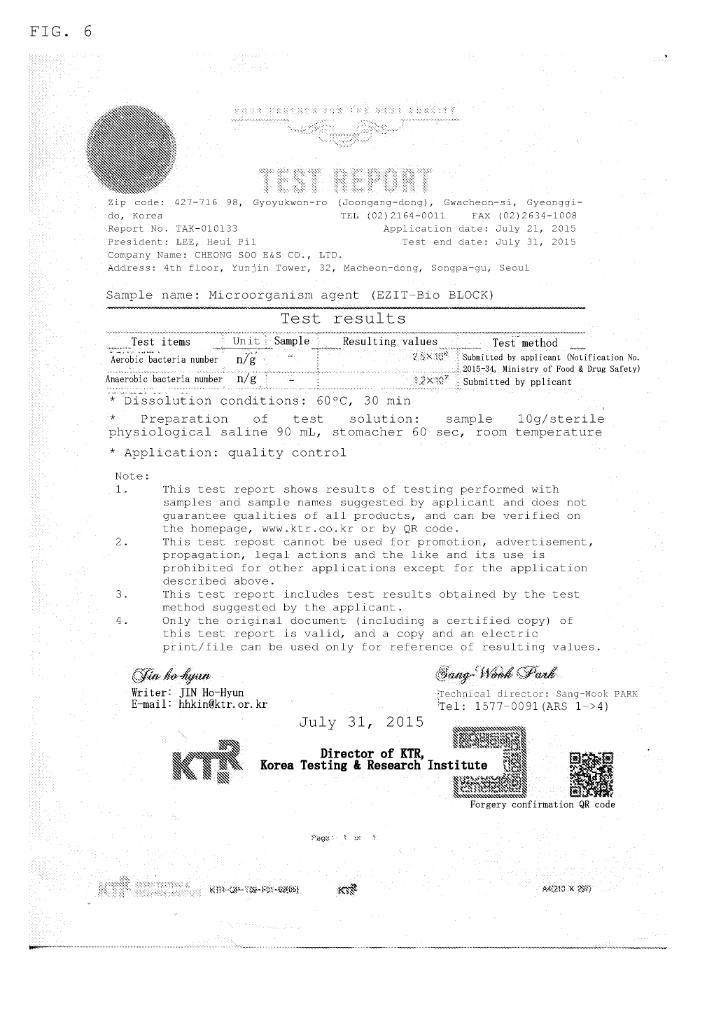

In the present test, a first prototype according to the embodiment of the present invention was produced according to Notification No. 2015-34 related to standards and regulations of food, implemented by the Ministry of Food & Drug Safety, 10 g of a sample obtained by powderizing the solid cleaning agent composition block was added to 90 mL of sterile physiological saline and homogenized in a stomacher (automated homogenizer) at room temperature for 60 seconds to prepare a test solution. The aerobic bacteria number and anaerobic bacteria number were measured. As shown in FIG. 5 annexed herein, measurement results showed that the aerobic bacteria number per 1 g of the test solution was 7.0×10⁹ (n/g) and the anaerobic bacteria number per 1 g of the test solution was 6.6×10⁸ (n/g).

The present test showed that the cleaning agent composition block according to the embodiment of the present invention contained the number of bacteria enough to perform potent cleaning and washing functions to degrade oils and fats under the condition of a very small particle size like a powder.

### TEST EXAMPLE 4

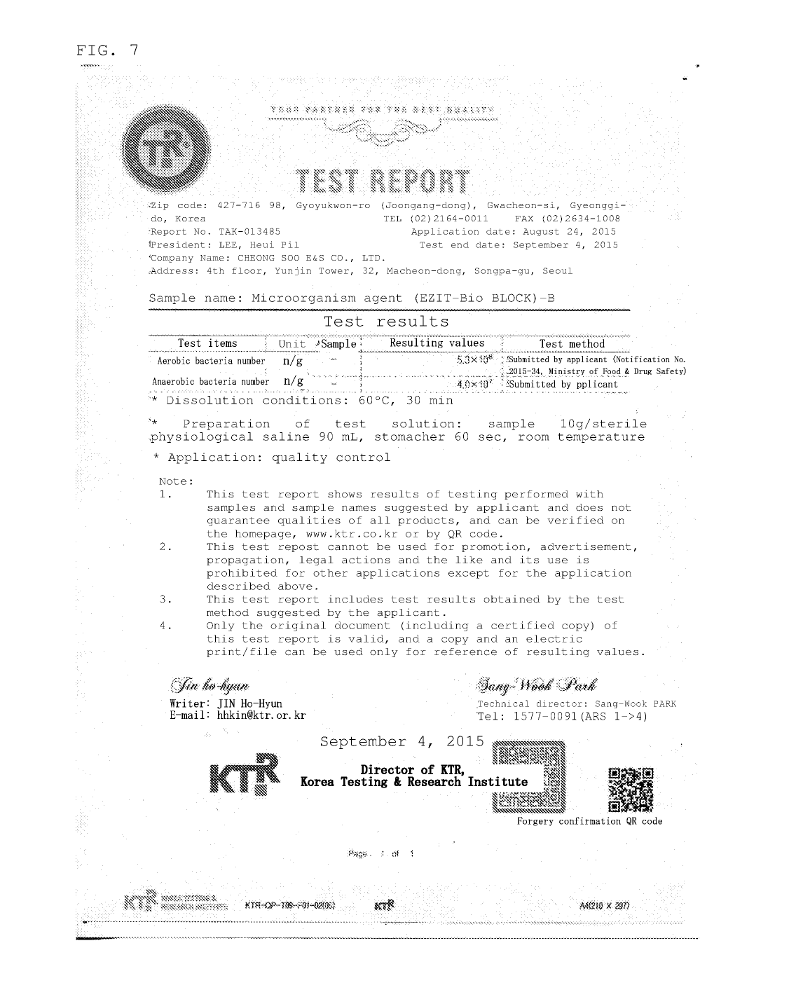

In the present test, a first prototype according to the embodiment of the present invention was produced according to Notification No. 2015-34 related to standards and regulations of food, implemented by the Ministry of Food & Drug Safety, 10 g of a solid cleaning agent composition block sample, which was a part of the prototype, was dissolved at a temperature of 60° C. for 30 minutes, added to 90 mL of a sterile physiological saline, and homogenized in a stomacher (automated homogenizer) at room temperature for seconds to prepare a test solution. Then, aerobic bacteria number and anaerobic bacteria number were measured. As shown in FIG. 6, measurement results showed that the aerobic bacteria number per 1 g of the test solution was 2.5×10⁸ (n/g) and the anaerobic bacteria number per 1 g of the test solution was 1.2×10⁷ (n/g).

The present test showed that the powdery cleaning agent composition block of Test Example 3 exhibited decreased aerobic and anaerobic bacteria numbers, whereas the solid cleaning agent composition block according to the present invention was accommodated in the drain cap equipped in an inlet of the drainpipe, was dissolved together with domestic wastewater and was flowed into the drainpipe, so that oils and fats adhered to the drainpipe were bio-degraded by powdery microorganisms, i.e., bacillus strains present in the cleaning agent composition block and a sufficient number of bacteria capable of performing cleaning and washing functions was present and acted.

### TEST EXAMPLE 5

In the present test, 500 kg of a first prototype according to the embodiment of the present invention was produced according to Notification No. 2015-34 related to standards and regulations of food, implemented by the Ministry of Food & Drug Safety, 10 g of a randomly selected solid cleaning agent composition block sample was dissolved at a temperature of 60° C. for 30 minutes, added to 90 mL of sterile physiological saline, and homogenized in a stomacher (automated homogenizer) at room temperature for 60 seconds to prepare a test solution. Then, aerobic bacteria number and anaerobic bacteria number were measured. As shown in FIG. 7, measurement results showed that the aerobic bacteria number per 1 g of the test solution was 5.3×10⁸ (n/g) and the anaerobic bacteria number per 1 g of the test solution was 4.0×10⁷ (n/g).

The present test showed that, like Test Example 4, the powdery cleaning agent composition block of Test Example 3 exhibited decreased aerobic and anaerobic bacteria numbers and detected a bacteria number two or more times that of Test Example 4. Accordingly, the solid cleaning agent composition block according to the embodiment of the present invention bio-degraded oils and fats, which had been accommodated in the drain cap, had been dissolved with domestic wastewater and had been adhered to the drainpipe, using powdery microorganisms, i.e., bacillus strains present in the cleaning agent composition block and a sufficient number of the bacteria was present so that they could actively perform cleaning and washing functions.

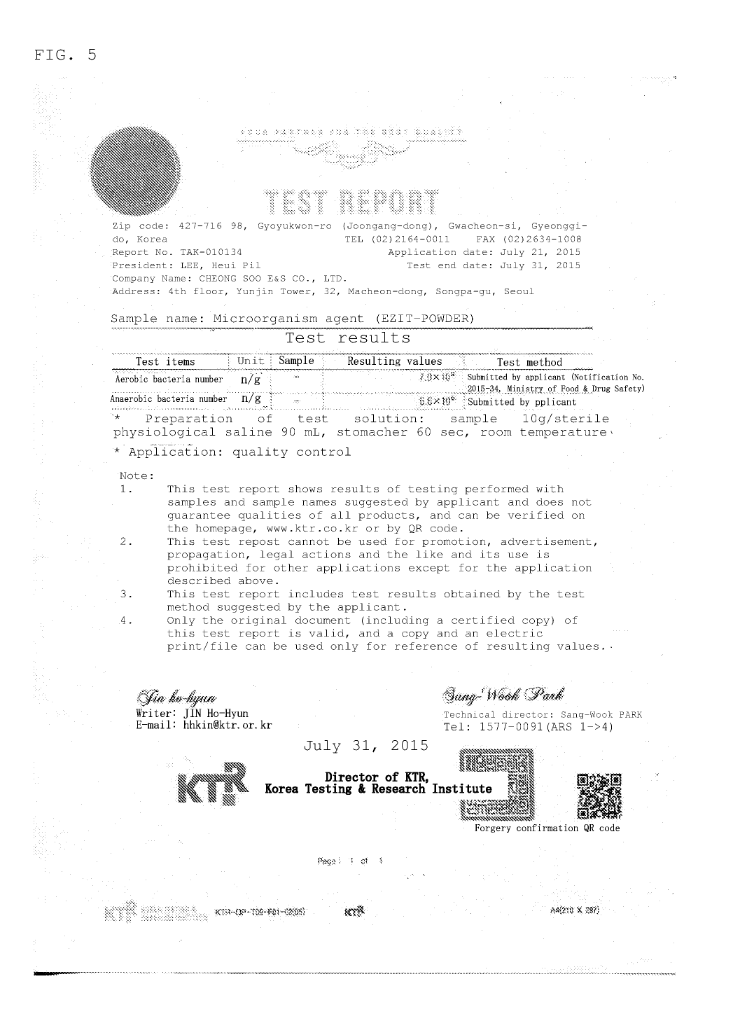

As apparent from Test Examples 1 to 5 described above, the microbial oil and fat cleaning agent composition block for drainpipes according to the embodiment of the present invention includes organic microorganisms, the bacillus strain, and can thus actively degrade oils and fats using the organic microorganisms, bacillus strain and is very effective in washing and cleaning inner walls of the drainpipe, or the inside of the water collector and water present inside the water collector through the organic microorganisms and a surfactant, as shown in the image of FIG. 4.

The microbial oil and fat cleaning agent composition block for drainpipes and the method of producing the same have excellent effects in that a solid microbial oil and fat cleaning agent composition block accommodated in a drain cap equipped in an inlet of a drainpipe is dissolved and introduced into the drainpipe so that oils and fats adhered to the drainpipe can be biodegraded by powdery microorganisms present in the microbial oil and fat cleaning agent composition block to maintain smooth flow of sewage, prevent oil and fats from adhering onto the drainpipe due to the cleaning agent and thus clean the inside of the drainpipe and remove odors, and the dissolved microbial oil and fat cleaning agent is discharged with sewage through the drainpipe and is collected in a water collector, and oils and fats adhered to the water collector are biodegraded by a microbial oil and fat cleaning agent to remove the odor inside the water collector, prevent adhesion of oils and fats to the water collector and thus maintain excellent water quality.

As apparent from the fore-going, the microbial oil and fat cleaning agent composition block for drainpipes according to the present invention is industrially applicable because the same product can be repeatedly produced in the same manner in the field of producing cleaning agents to produce solid microbial oil and fat cleaning agents accommodated in the drain cap equipped in the inlet of the drainpipe.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of producing a microbial oil and fat cleaning agent composition block for drainpipes, the method comprising:
elevating a temperature of a reaction tank to 85° C. by heating (S1);
adding a non-ionic surfactant to the heated reaction tank and stirring the same to completely dissolve the non-ionic surfactant (S2);
adding a linear alkyl benzene-based surfactant to the completely dissolved non-ionic surfactant and stirring the same until the linear alkyl benzene-based surfactant is completely dissolved (S3);
secondarily adding a linear alkyl benzene-based surfactant and stirring the same until the linear alkyl benzene-based surfactant is completely dissolved while maintaining a temperature at 80° C. or less (S4);
adding a non-ionic higher alcohol and stirring the same while maintaining a temperature at 75° C. (S5);
adding a cleaning agent and stirring the same while maintaining a temperature at 75 to 76° C. (S6);
adding a dye, stirring the same while maintaining a temperature at 65 to 70° C. and cooling the same (S7);
adding a dissolution controller and stirring the same (S8);
adding an organic microorganism powder and stirring the same while maintaining a temperature at 65 to 70° C. (S9);
adding a thickener and a defoamer and stirring the same (S10); and
filling a die with the composition mixed in the previous steps (S11).

2. The method according to claim 1, wherein the organic microorganism powder added in S9 comprises bacillus strain.

3. A microbial oil and fat cleaning agent composition block for drainpipes produced by the method according to claim 1, comprising:
31 to 34% by weight of a non-ionic surfactant;
29 to 33% by weight of a linear alkyl benzene-based surfactant;
15 to 17% by weight of a non-ionic higher alcohol;
7 to 9% by weight of a cleaning agent;
0.1% by weight of a dye;
4.5 to 5.5% by weight of a dissolution controller;
2.5 to 3.5% by weight of an organic microorganism powder;
3.5 to 4.5% by weight of a thickener; and
2.5 to 3.5% by weight of a defoamer, based on the total weight of the microbial oil and fat cleaning agent for drainpipes.

4. The microbial oil and fat cleaning agent composition block according to claim 3, wherein the organic microorganism powder comprises bacillus strain.
